(19) United States
(12) Patent Application Publication
(10) Pub. No.: US 2020/0345911 A1
(43) Pub. Date: Nov. 5, 2020

(54) PREDICTIVE QRS DETECTION AND R-TO-R TIMING SYSTEMS AND METHODS

(71) Applicant: NuPulseCV, Inc., Raleigh, NC (US)

(72) Inventors: Robert M. Smith, Raleigh, NC (US); Nagarjuna Reddy Chinta, Apex, NC (US); Ryan Daniel McCormick, Durham, NC (US); Douglas Edward Ivers, Cary, NC (US)

(21) Appl. No.: 16/401,407

(22) Filed: May 2, 2019

(51) Int. Cl. A61M 1/10; A61B 5/04; A61B 5/0456; G16H 50/20

(52) U.S. Cl. CPC A61M 1/1086; A61B 5/04014; A61M 2230/04; A61M 1/1072; G16H 50/20; A61B 5/0456

(57) ABSTRACT

The present disclosure is directed towards systems and methods built for predictively timing the inflation and/or deflation of an intra-aortic balloon pump. A controller operates in three states: (1) initialization state, (2) learning state, and (3) peak detection state. The controller decomposes a patient's electrocardiogram signal to a power signal. It then learns characteristics of the patient's electrocardiogram signal during the learning state and computes adaptive threshold parameter values. During the peak detection state, the controller applies the learnt threshold parameter values on a current electrocardiogram signal to identify occurrence and timings of R peaks in the electrocardiogram signal. The R-to-R peak timings are then used to trigger inflation of an intra-aortic balloon pump.

### BACKGROUND

The invention relates generally to medical devices, and more particularly to systems and methods related to the operation of medical devices, such as intra-aortic balloon pumps, based on physiological measurements. About 5.7 million adults in the United States have heart failure, according to the U.S. Centers for Disease Control and Prevention website. Each year, about 100,000 people nationally are diagnosed with advanced heart failure and require some sort of mechanical support, such as intra-aortic balloon pumps. The balloon pump is positioned inside the aorta, typically in the proximal descending aorta. The balloon pump (typically 40-50 milliliters in capacity) is inflated and deflated in time with the contraction of the left ventricle. During diastole, the balloon is inflated, thereby driving blood in the ascending aorta and aortic arch into the coronary arteries to supply oxygen to the heart muscle. During systole, as the left ventricle contracts, the balloon is deflated so as to decrease the afterload. This procedure is termed “counterpulsation.”

### BRIEF DESCRIPTION OF THE DRAWINGS

The accompanying drawings, which are incorporated in and constitute a part of this specification, illustrate several embodiments and, together with the description, serve to explain the disclosed principles. In the drawings:

FIGS. 1A and 1B illustrate an intra-aortic balloon pump in accordance with some embodiments.

FIG. 2 is a block diagram showing some of the components typically incorporated in a controller in accordance with some embodiments.

FIG. 3A illustrates components of a controller in accordance with some embodiments.

FIG. 3B illustrates a state diagram of the controller in accordance with some embodiments.

FIG. 4A illustrates a QRS complex in an electrocardiogram signal in accordance with some embodiments.

FIG. 4B is a flow diagram illustrating a method performed by a power signal module of a controller in accordance with some embodiments.

FIGS. 4C and 4D illustrate an electrocardiogram signal received and processed in accordance with some embodiments.

FIG. 4E illustrates a transformation signal in accordance with some embodiments.

FIGS. 4F and 4G illustrate decomposed electrocardiogram signals in accordance with some embodiments.

FIGS. 4H and 4I illustrate electrocardiogram signals and corresponding decomposed power signals in accordance with some embodiments.

FIG. 5A is a flow diagram illustrating a method performed by a QRS Detection module of a controller in accordance with some embodiments.

FIG. 5B illustrates the selection of h-peak values to compute threshold values in accordance with some embodiments.

FIGS. 6 and 7 illustrate the result of applying an adaptive threshold to detect the occurrence of R peaks in accordance with some embodiments.

### DETAILED DESCRIPTION

Intra-aortic balloon pump devices typically process ECG signals for efficient and optimum operation. One of the most important parts of ECG signal processing and intra-aortic balloon pump operation is interpretation of QRS complex and obtaining its characteristics. The QRS complex is a name for the combination of three of the graphical deflections seen on a typical electrocardiogram. In adults, the QRS complex normally lasts 0.06-0.10 seconds; in children and during physical activity, it may be shorter. The Q, R, and S waves occur in rapid succession, do not all appear in all leads, and reflect a single event and thus are usually considered together. A Q wave is any downward deflection immediately following the P wave. An R wave follows as an upward deflection, and the S wave is any downward deflection after the R wave. The T wave follows the S wave, and in some cases, an additional U wave follows the T wave.

R wave is one of the most important sections of this complex, which has an essential role in diagnosis of heart rhythm irregularities and also in determining heart rate variability (HRV). Traditional systems for detecting QRS complex include differentiation methods, digital filters, neural networks, filter banks, hidden Markov models, genetic algorithm, and maximum a posterior (MAP) estimator. These methods are highly sensitivity to noise and generally cause errors in detecting the correct timing of R waves in ECG signals. As a result, conventional systems and methods for intra-aortic balloon pumps suffer from an inability to predictively time the inflation and/or deflation of the balloon pump.

In view of the shortcomings of conventional approaches to accurately detecting the occurrence and timing R waves, the inventors have recognized that a new approach to predicting R wave and R peak timings that is more accurate, reliable, and efficient would have significant utility.

A software, hardware, and/or firmware facility (“the facility”) that provides a solution to predictively time the occurrence of future R peaks to efficiently inflate and/or deflate an intra-aortic balloon pump is described. In several embodiments, the facility operates as a state machine comprising three states: (1) initialization state, (2) learning state, and (3) peak detection state. By performing in some or all of the manners discussed below, the facility improves the prediction of future R-peaks so that a corresponding intra-aortic balloon pump is inflated and/or deflated in an efficient and reliable manner. This results in an improved patient care experience that keeps a patient's heart pumping in a closely natural rhythm. The facility described herein for QRS detection and/or R-to-R timing may be used for various devices including, but not limited to pacemakers, heart monitors, defibrillators, heartrate monitors, smart watches, athletic accessories, and the like.

### Description of Figures

The following description provides certain specific details of the illustrated examples. One skilled in the relevant art will understand, however, that embodiments can be practiced without many of these details. Likewise, one skilled in the relevant art will also understand that the present disclosure can include many other features not described in detail herein. Additionally, some well-known structures or functions may not be shown or described in detail below, to avoid unnecessarily obscuring the relevant descriptions of the various examples.

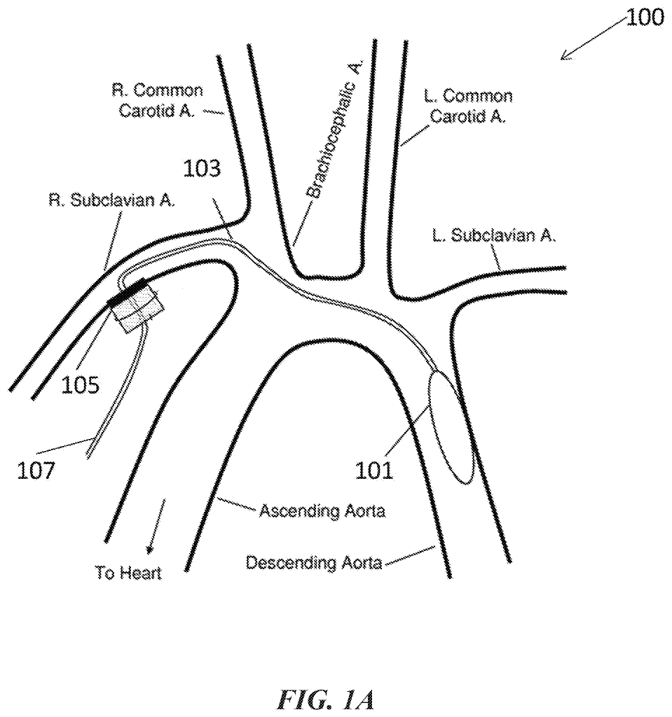

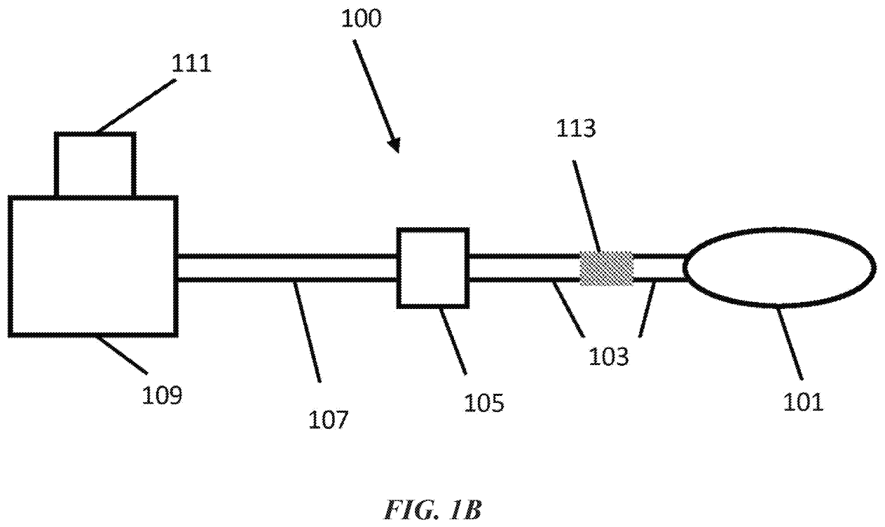

Several implementations are discussed below in more detail in reference to the figures. Turning now to the figures, FIGS. 1A and 1B illustrate an example of an intra-aortic balloon pump facility 100. The intra-aortic balloon pump 100 may be sized and shaped to dangle inside a patient's aorta. The intra-aortic balloon pump 100 may include a balloon 101 that is configurably connected to an internal drive line 103 that connects to a skin interface device 105. As illustrated in FIG. 1B, an external drive line 107 may connect the skin interface device 105 to a driver 109.

At its proximal end, balloon 101 is connected to the distal end of the internal drive line 103. The skin interface device 105 connects the proximal end of the internal drive line 103 to the distal end of the external drive line 107. The proximal end of the external drive line 107 is connected to the driver 109. The driver 109 may comprise or be connected to a controller 111. An arterial interface 113 may be sized and shaped to pass the internal drive line 103 through an arterial wall.

In several embodiments, the intra-aortic balloon pump 100 may include a controller 111 configured in accordance with the systems and methods described herein. The controller 111 may control the operation of the valves and bellows (not shown) of the driver 109 to control the flow of a pumping medium (for example, air) and the inflation and deflation of the balloon 101. The controller 111 may receive one or more signals from the balloon 101 and surrounding areas. Signals may include those received from electrodes, pressure sensors and the like (for example, electrocardiogram signals). In several embodiments, the controller 111 receives one or more of the following data values about a patient from, for example, the skin interface device 105: patient identifier (for example, a unique patient identification alphanumeric string), name of the patient, age of the patient, average heart rate of the patient, maximum heart rate of the patient, minimum heart rate of the patient, pulse of the patient, average R-to-R time interval of the patient, maximum R-to-R time interval of the patient, minimum R-to-R time interval of the patient, average R-peak of the patient, maximum R-peak of the patient, minimum R-peak of the patient, and so on. In several embodiments, controller 111 retrieves and/or computes one or more of the above listed data values based on a subset of the received information about the patient. For example, based on a patient identifier, controller 111 retrieves the following data values (from, for example, a communicatively connected memory): name of the patient, average heart rate of the patient, pulse of the patient, and average R-to-R time interval of the patient.

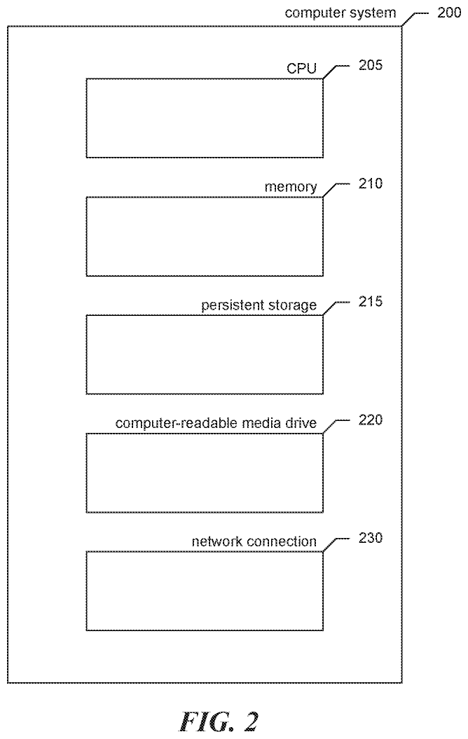

FIG. 2 is a block diagram showing some of the components typically incorporated in controller 111. In various embodiments, the controller 111 include zero or more of each of the following: a central processing unit (“CPU”) 101 for executing computer programs; a computer memory 102 for storing programs and data while they are being used, including the facility and associated data, an operating system including a kernel, and device drivers; a persistent storage device 103, such as a hard drive or flash drive for persistently storing programs and data (for example, patient related data); a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. For example, via the network connection 105, controller 111 can retrieve patient related data from a remote data storage location (for example, a cloud data storage location). While computer systems configured as described above are typically used to support the operation of the controller 111, those skilled in the art will appreciate that the facility may be implemented using devices of various types and configurations, and having various components.

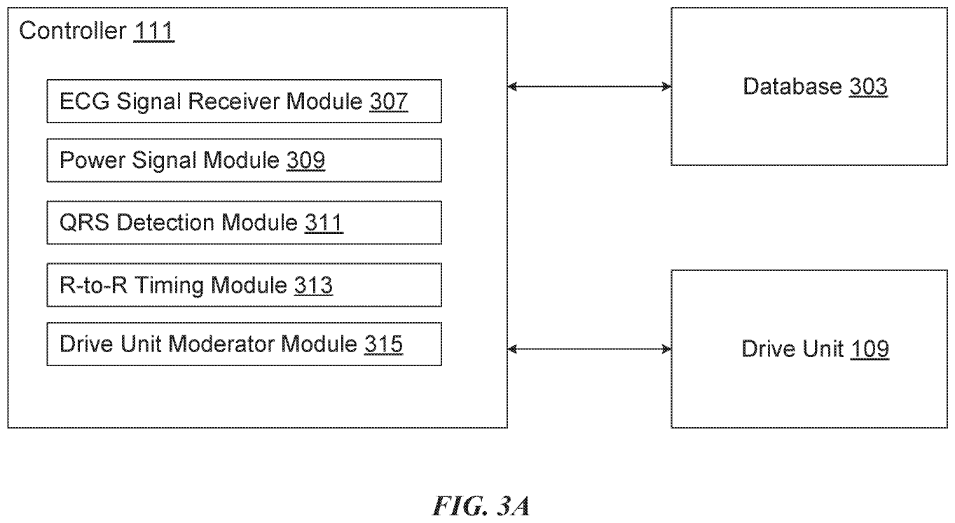

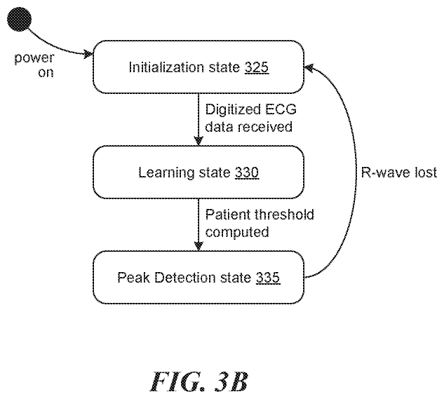

FIG. 3A illustrates components of a controller in accordance with some embodiments. In some embodiments, the controller may be configured to be on a drive unit for an intra-aortic balloon pump. Controller 111 may include signal receiver module 307, power signal module 309, QRS detection module 311, R-to-R timing module 313, and drive unit moderator module 315. In several embodiments, the controller operates as a state machine comprising three states as depicted in FIG. 3B: (1) initialization state 325, (2) learning state 330, and (3) peak detection state 335.

### Initialization State

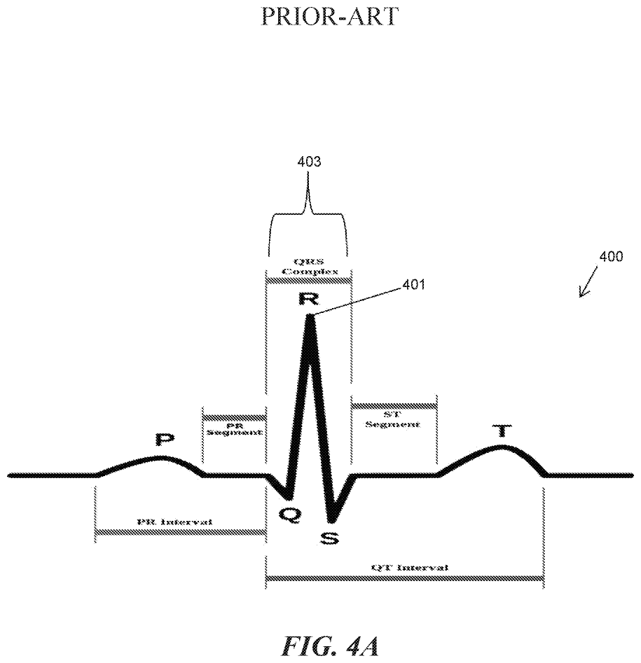

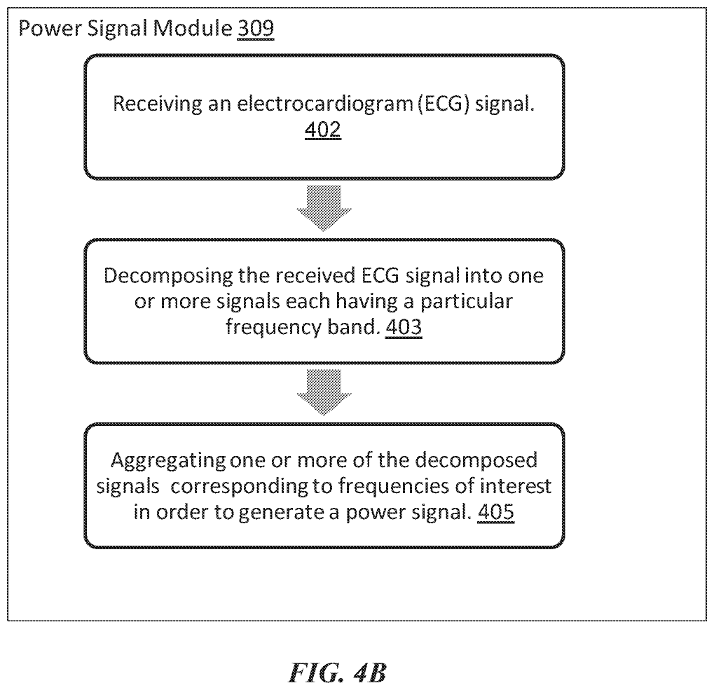

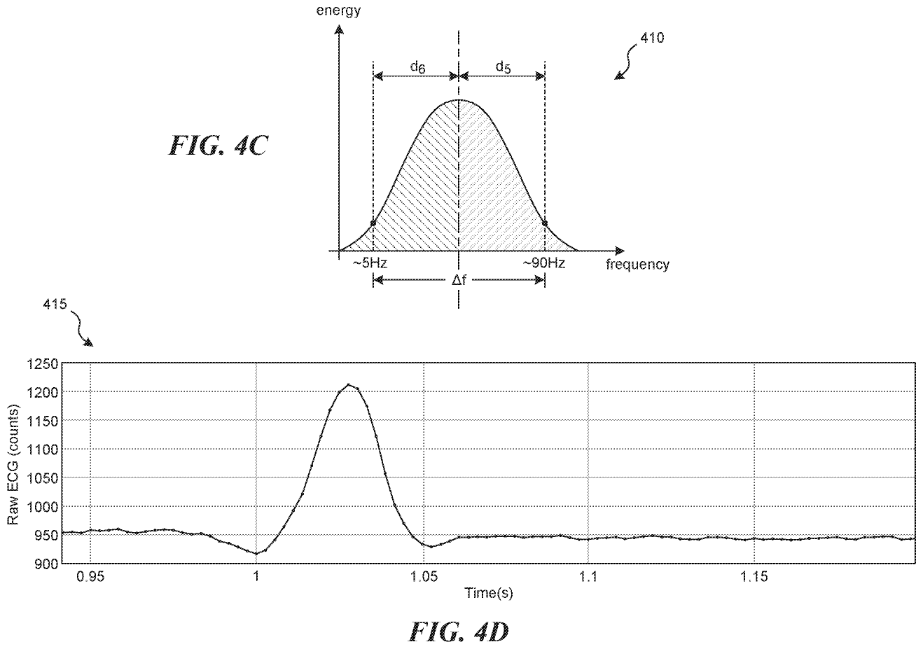

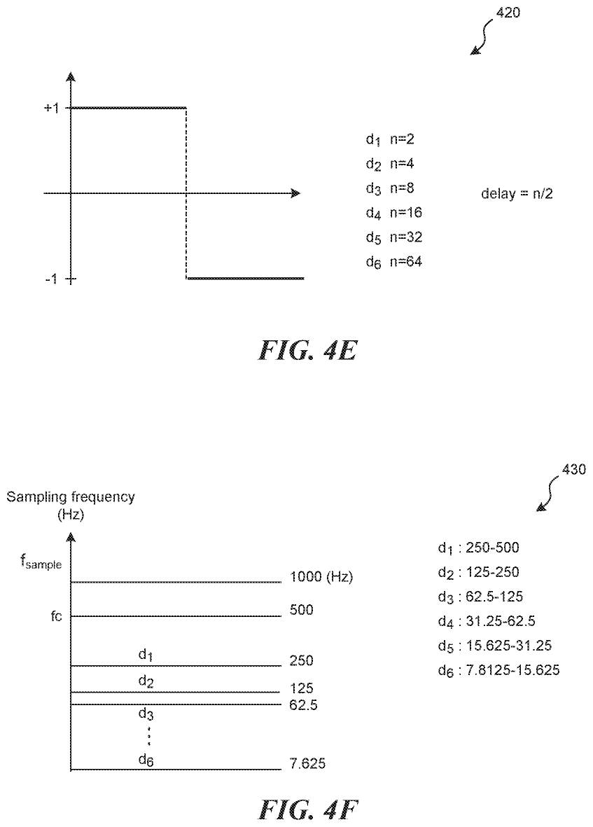

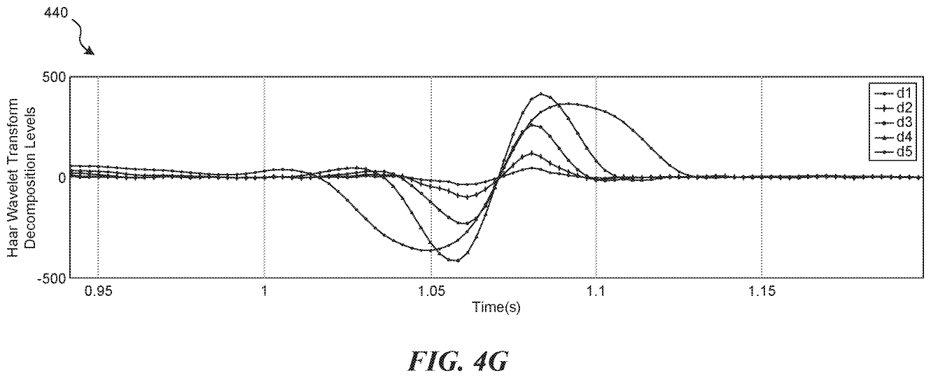

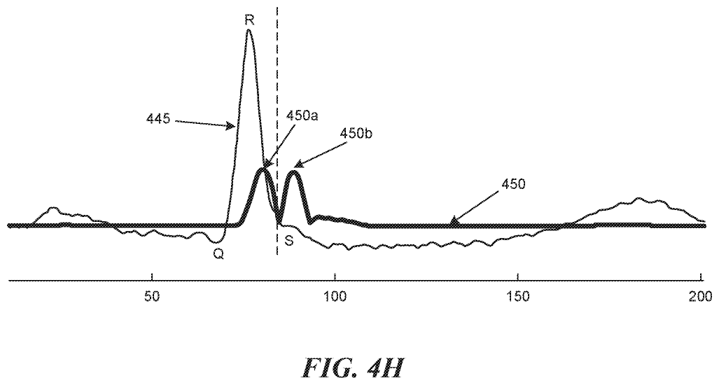

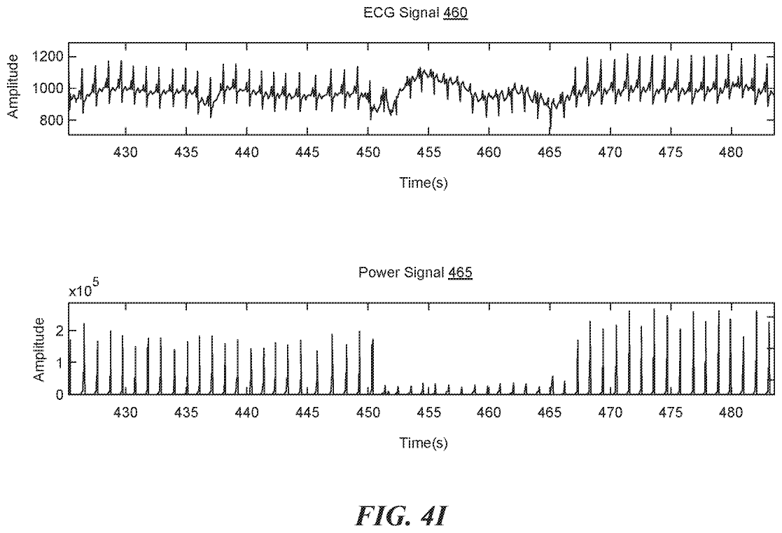

The signal receiver module 307 of the controller may be configured to receive a signal (for example, analog electrocardiogram signal). For example, the controller receives an electrocardiogram signal from remote devices (for example, one or more skin interface devices (SID) implanted in a patient's body). Applicants' U.S. Pat. No. 9,265,871 and U.S. Pat. No. 10,137,230 provide more details regarding suitable SIDs and are incorporated by reference herein in their entireties. In several embodiments, the signal receiver module 307 digitizes the received electrocardiogram signal and/or applies one or more filters to obtain a filtered electrocardiogram signal that is suitable for further processing by the facility. Example filters may include, but are not limited to, bandpass filters, derivative filters, squaring filters, and/or applying moving window integrals. FIG. 4A illustrates a portion of an electrocardiogram signal 400 (including a QRS complex 403) in accordance with some embodiments. A QRS complex 403 is a feature of the electrocardiogram signal indicative of depolarization of a heart. An R-peak 401 is an element within the QRS complex 403. Real-time detection of these peaks requires an appropriate threshold that is capable of differentiating between significant peaks, which typically denote the QRS complex, and minor peaks which may be associated with noise, T waves, or P waves. The facility described herein is configured to accurately detect and predict QRS complexes and R-peaks. During the initialization state 325, the controller receives the electrocardiogram signal data and initializes one or more variables, buffers, memory, and the like. For example, the controller receives a patient identifier (for example, a unique patient identification alphanumeric string) to initialize values of one or more of the following variables: name of the patient, age of the patient, average heart rate of the patient, maximum heart rate of the patient, minimum heart rate of the patient, pulse of the patient, average R-to-R time interval of the patient, maximum R-to-R time interval of the patient, minimum R-to-R time interval of the patient, average R-peak of the patient, maximum R-peak of the patient, minimum R-peak of the patient, and so on.

### Learning State

After completing the initialization state 325, the controller enters the learning state 330. During the learning state 330, the controller generates a first power spectrum signal by decomposing the received first electrocardiogram signal (for example, using historical electrocardiogram signal for a patient). The power signal module 309 in the controller is configured to generate a power signal based on the digitized and/or filtered electrocardiogram signal. FIG. 4B is a flow diagram illustrating a method performed by the power signal module 309 of the controller 111 in accordance with several embodiments. The method includes the acts of receiving an electrocardiogram (ECG) signal 401 (act 402). FIGS. 4C and 4D illustrate portions of electrocardiogram signals 410 and 415 received and processed in accordance with some embodiments. At act 403, the power signal module 309 may decompose the received electrocardiogram signal into one or more sub-bands or frequencies of interest. At act 405, the power signal module 309 may then aggregate one or more sub-bands of interest to generate the power signal. The one or more sub-bands of interest may be selected based on characteristics of the received signal, such as the number of received information vectors, number of electrode leads used to capture the electrocardiogram signal, location of electrode leads used to capture the electrocardiogram signal, and so on.

As an illustrative example, the facility collects electrocardiogram signal data using three electrode leads (corresponding to three vectors each) and chooses data from one of the electrode leads (corresponding to one of the three vectors) for monitoring at 1000 Hz sampling. The power signal module 309 applies a transformation signal (for example, a Haar wavelet 420 illustrated in FIG. 4E) to decompose the electrocardiogram signal from the selected electrode lead into discrete frequency signals. For example, FIGS. 4F and 4G illustrate decomposed electrocardiogram signals 430 and 440 in accordance with some embodiments. The power signal module 309 then selects the following sub-bands of interest: a first portion in the d5 frequency range (15.625 Hz to 31.25 Hz) and a second portion in the d6 frequency range (7.8125 Hz to 15.625 Hz). The power signal module 309 aligns the selected portions in the d5 and d6 frequency range to compute the power signal (“h-signal”)=|d5.d6|.

In several embodiments, the facility collects electrocardiogram signal data using three (or more) electrode leads (corresponding to three (or more) vectors each) and chooses data from all three electrode leads (all three vectors) for monitoring at 500 Hz sampling. After applying the Haar wavelet transform on the electrocardiogram data, the power signal module 309 selects the following sub-bands of interest from the vector with the best signal characteristics: a first portion in the d4 frequency range (31.25 Hz to 62.5 Hz) and a second portion in the d5 frequency range (15.625 Hz to 31.25 Hz). The delay in detecting and processing the electrocardiogram data is a direct function of the number of electrodes (or vectors) whose data is utilized (delay=n/2, where n is a function of the number of the higher frequency band). For example, when data from a single electrode (and a single vector) is used by the power signal module 309 so that the higher frequency band selected is d6,

$$the amount of delay = 32 seconds ( \frac{{2}^{6}}{2} ) .$$

Similarly, when data from three electrodes (and three vectors) is used by the power signal module 309 so that the higher frequency band selected is 5,

$$the amount of delay = 16 seconds ( \frac{{2}^{5}}{2} ) .$$

In several embodiments, the power signal includes two peaks that correspond to the rise from Q to R and the fall from R to S in the QRS complex. For example, FIG. 4H illustrates a power signal 450 that includes two peaks 450a and 450b that correspond to the rise from Q to R and the fall from R to S in the QRS complex of a corresponding electrocardiogram signal 445. FIG. 4I is another illustration that depicts an electrocardiogram signal 460 and its corresponding decomposed signal 465.

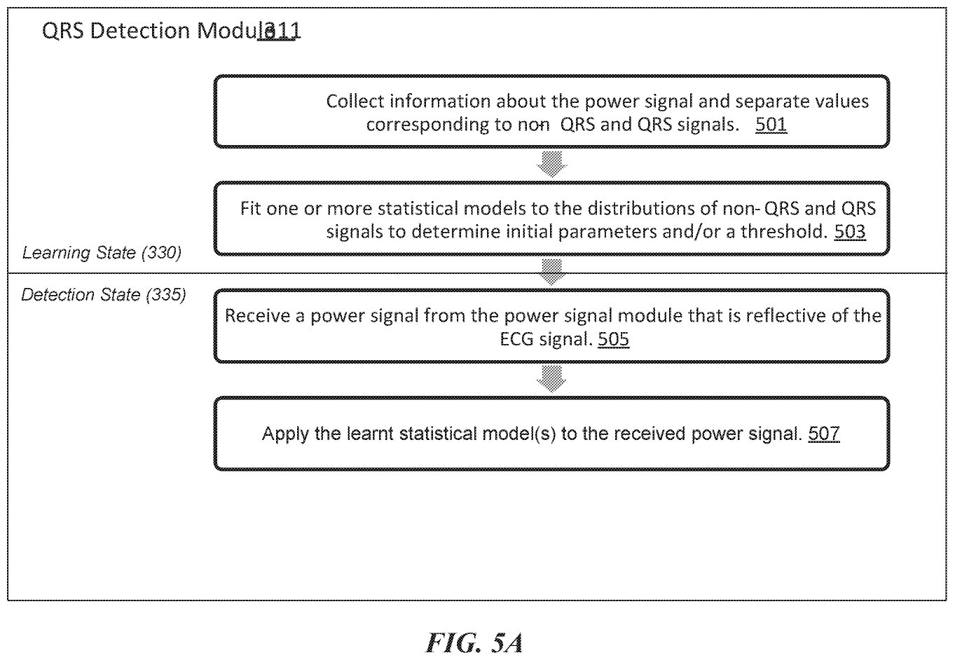

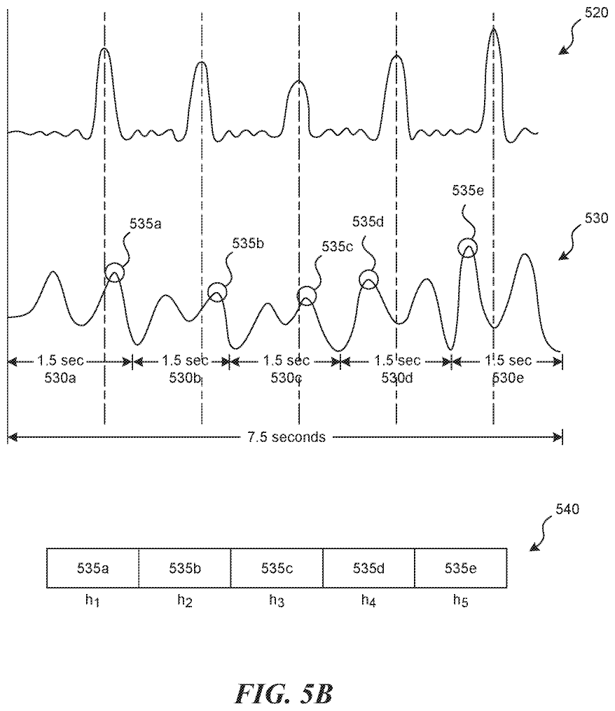

In several embodiments, after generating a power signal corresponding to the received electrocardiogram signal, the QRS detection module 311 in the controller may be trained on attributes derived from the generated power signal to detect future R peaks. FIG. 5A is a flow diagram illustrating a method performed by the QRS Detection module 311 of a controller in accordance with some embodiments. At act 501, the QRS Detection module 311 collects information about the computed power signal and separates values corresponding to non-QRS and QRS signals. At act 503, the QRS Detection module 311 fits one or more models to the distribution of non-QRS and QRS signals to determine initial parameters and/or one or more threshold values. For example, during the learning state of the controller, the QRS detection module 311 of the controller selects a first portion of the generated power spectrum signal and divides it into one or more subsets comprising sub-portions of the selected portion of the generated power spectrum signal.

As an example, illustrated in FIG. 5B, the controller selects a portion spanning 7.5 seconds of the power spectrum signal 530 corresponding to an electrocardiogram signal 520. The controller divides the power signal 530 into five subsets (530a, 530b, 530c, 530d, and 530e), each spanning 1.5 seconds. For each of these subsets, the controller computes a h-peak value of the sub-portion. In several embodiments, the controller stores the computed h-peak value of the selected portion of the power spectrum signal falling within the subset (that is, maximum h-signal value per subset). For example, the controller stores the h-peak values of the subset (535a, 535b, 535c, 535d, and 535e) in a first threshold buffer 540. The controller then computes a first adaptive threshold R-peak value based on the computed peak values stored in the first threshold buffer. For example, the controller computes a median value (or mean, weighted mean, weighted median, and so on) for the h-peak values in the threshold buffer 540 and then computes a percentage (for example, a configurable percentage of 40%) of the median value as the first adaptive threshold R-peak value for the patient. The configurable percentage can be a value within a predetermined range (for example, 20% to 27%). The configurable percentage value can be configured (or reconfigured) automatically or manually (for example, by a physician). For example, the configurable percentage value can be configured (or reconfigured) automatically using a Signal-to-Noise Ratio (SNR) of the ECG Signal. As the SNR increases, the configurable percentage value can be reduced, which leads to faster detections. On the other hand, when the patient's noise level rises to the point where it almost drowns out the R-waves, the configurable percentage value can be raised to avoid triggering the pump on noise. As another example, the configurable percentage value can be configured (or reconfigured) automatically using a measure of the R-Wave Amplitude Variability. When a patient's R-wave peak amplitude varies widely and often—as may be the case with heart failure patients with premature ventricular contractions (PVCs), bigeminy or trigeminy, or other cardiac pathologies—the controller can adjust the configurable percentage value so that it can detect both low-amplitude and high-amplitude R-waves. An example of such a measure might be the standard deviation or variance of some set of maximum h-signal peak values measured during the learning phase, or even during a separate “check-up” time period of a particular duration every so often while running in the detection phase.

### Peak Detection State

After computing the first adaptive threshold R-peak value for the patient, the controller enters the peak detection state 335. As depicted in FIG. 5A at act 505, the QRS Detection Module 311 of the controller receives a patient's current/most recent electrocardiogram signal (second electrocardiogram signal). Like the learning state 330, the controller in the peak detection state 335 generates a second power spectrum signal by decomposing the received second electrocardiogram signal. For example, the controller selects a first portion of the generated second power spectrum signal and divides the selected first portion of the generated second power spectrum signal into one or more second subsets comprising sub-portions of the selected first portion of the generated second power spectrum signal. For example, the controller selects a portion spanning 7.5 seconds of the second power spectrum signal and divides it into 5 subsets, each spanning 1.5 seconds. For each of these subsets, the controller computes a peak value of the sub-portion of the selected first portion of the second power spectrum signal falling within the subset (that is, a peak h-signal value per subset). In several embodiments, the controller stores the computed peak value of the subset in a second threshold buffer. The controller then computes a second adaptive threshold R-peak value based on the computed peak values stored in the second threshold buffer. For example, the controller computes a median value for the h-signal values in the second threshold buffer and then computes a percentage (for example, a configurable percentage of 40%) of the median value as the second adaptive threshold R-peak value for the patient.

At act 507, the controller applies the model(s) learnt during the learning state (for example, at act 503) to the second power spectrum signal. In several embodiments, the controller determines whether a currently received electrocardiogram signal includes a QRS complex by applying the first adaptive threshold R-peak value, the second adaptive threshold R-peak value, or both to a power signal corresponding to the currently received electrocardiogram signal. For example, in some embodiments, the presence of the QRS complex is determined when the power signal is greater than first adaptive threshold R-peak value, the second adaptive threshold R-peak value, or both. The absence of the QRS complex is determined when the power signal less than the threshold value(s). Since the electrocardiogram signal is a non-stationary signal whose statistical characteristics change over time, in some embodiments, approaches have been developed to make this threshold adaptive such that it changes with changing characteristics of the signal. Additionally, the facility may include other mechanisms to improve detection consistency, such as including prior information about the location of QRS, placing limits on how close adjacent QRS complexes can be in time, and improving future decisions based on past errors in detection.

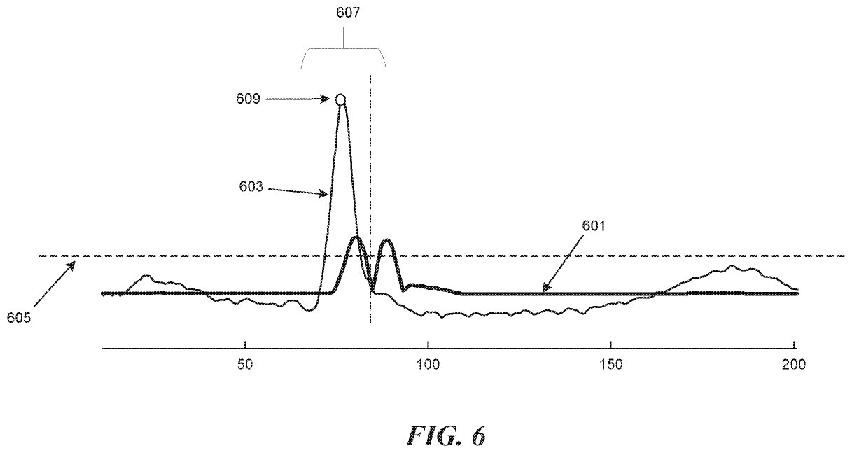

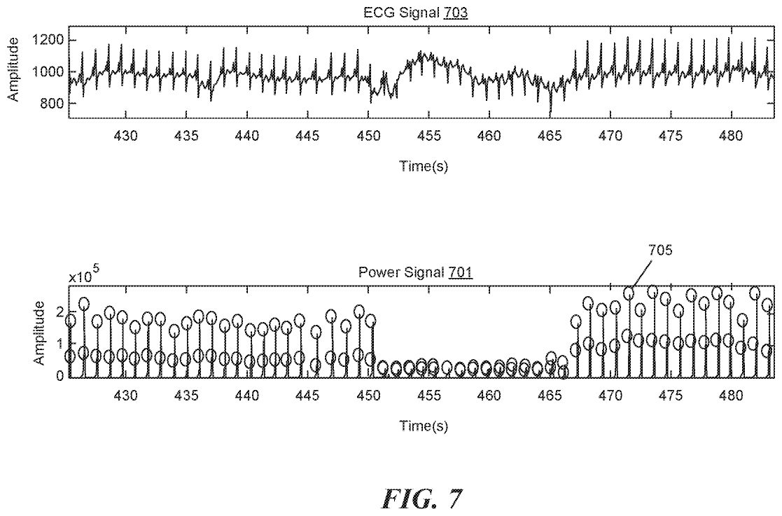

FIGS. 6 and 7 illustrate the result of applying an adaptive threshold to detect the occurrence of R peaks. For example, as illustrated in FIG. 6, a power signal 601 may be generated that is reflective of the electrocardiogram signal 603. An adaptive threshold 605 may be applied to the power signal 601 to detect a QRS complex 607 and estimate an R-peak 609. FIG. 7 similarly illustrates the result of applying an adaptive threshold to detect the occurrence of R peaks. As illustrated a power spectrum signal 701 may be generated that is reflective of the electrocardiogram signal 703. As illustrated, R-peak detections 705 may be overlaid upon the power spectrum 701.

When it is determined that the received electrocardiogram signal includes the QRS complex, QRS signals, QRS detections, R-peaks, and other data from the QRS detection module 311 may be passed to the R-to-R timing module 313. The R-to-R timing module 313 computes an R-to-R time interval value indicative of timing between two successive R-peaks in successive QRS complexes in the received second electrocardiogram signal of the patient. The R-to-R time interval value may be determined by taking the average of a buffer of the 7 most recent R-R intervals. In some embodiments, the R-to-R time interval value is calculated as the median of the 5 most recent R-to-R intervals. Using the median may result in superior outlier rejection of the median (versus the mean) along with the faster response of the system to rapidly changing heart rates. The controller may determine the patient's heart rate is by taking the reciprocal of the calculated R-R interval and multiplying by 60 to yield a heart rate in beats per minute (BPM). The controller may also generate a prediction of timing of one or more future R-peaks based on the computed R-to-R time interval value indicative of timing between two successive R-peaks in successive QRS complexes. In several embodiments, the controller triggers an inflation of the at least one intra-aortic balloon pump based on the generated prediction of timing of the future R-peak(s).

Based on the prediction of an occurrence of a future R element, the drive unit moderator 315 of the controller 111 may be used to control the drive unit 305 of an intra-aortic balloon pump or other medical device. For example, the inflation of the balloon may be timed to the predicted occurrence of a future R element in the QRS complex.

The techniques described herein have several advantages over conventional methods because they are initialized quickly by approximating characteristics of QRS and non-QRS distributions rather than building them directly. Additionally, they require less memory and processing than conventional systems, due to approximating the QRS and non-QRS distributions rather than calculating them directly. Furthermore, the described techniques for the QRS detection module 311 are faster in adapting to sudden changes in the power signal.

While illustrative embodiments have been described herein, the scope thereof includes any and all embodiments having equivalent elements, modifications, omissions, combinations (e.g., of aspects across various embodiments), adaptations and/or alterations as would be appreciated by those in the art based on the present disclosure. For example, the number and orientation of components shown in the exemplary systems may be modified.

Those skilled in the art will appreciate that the process shown in FIGS. 4B, 5A, 5B, and 8 may be altered in a variety of ways. For example, the order of the acts may be rearranged; some acts may be performed in parallel; shown acts may be omitted, or other acts may be included; a shown act may be divided into sub acts, or multiple shown acts may be combined into a single act, etc.

It will be appreciated by those skilled in the art that the above-described facility may be straightforwardly adapted or extended in various ways. While the foregoing description makes reference to particular embodiments, the scope of the invention is defined solely by the claims that follow and the elements recited therein.

What is claimed is:

1. A system for operating intra-aortic balloon pumps, the system comprising:
at least one intra-aortic balloon pump placed in the proximity of a heart of a patient; and
at least one controller configurably connected to the at least one intra-aortic balloon pump, wherein the at least one controller comprises at least one processor and at least one non-transitory memory, wherein the at least one controller is configured to:
initialize values of one or more parameters based on an identity of the patient;
receive a first electrocardiogram signal of the patient;
generate a first power spectrum signal by decomposing the received first electrocardiogram signal, wherein the first power spectrum signal is generated by:
applying at least one transformation signal to decompose the received first electrocardiogram signal into discrete frequency signals;
selecting one or more portions of the discrete frequency signals; and
computing the first power spectrum signal by aligning the selected one or more portions of discrete frequency signals,
wherein the computed first power spectrum signal is based on a first selected portion of the discrete frequency signals and a second selected portion of the discrete frequency signals,
wherein the first selected portion of the discrete frequency signals is in a first frequency range of 15.625 Hz to 31.25 Hz, and
wherein the second selected portion of the discrete frequency signals is in a second frequency range of 7.8125 Hz to 15.625 Hz;
select a first portion of the generated first power spectrum signal;
divide the selected first portion of the generated first power spectrum signal into one or more subsets comprising sub-portions of the selected first portion of the generated first power spectrum signal;
for each subset in the one or more subsets of the selected first portion of the generated first power spectrum signal:
compute a peak value of the sub-portion of the selected first portion of the first power spectrum signal falling within the subset; and
store the computed peak value of the subset in a first threshold buffer; and
compute a first adaptive threshold R-peak value based on the computed peak values stored in the first threshold buffer.

2. The system of claim 1 wherein the at least one controller is further configured to:
receive a second electrocardiogram signal of the patient;
generate a second power spectrum signal by decomposing the received second electrocardiogram signal, wherein the second power spectrum signal is generated by:
applying at least one transformation signal to decompose the received second electrocardiogram signal into second discrete frequency signals;
selecting one or more portions of the second discrete frequency signals; and
computing the second power spectrum signal by aligning the selected one or more portions of the second discrete frequency signals,
wherein the computed second power spectrum signal is based on a first selected portion of the second discrete frequency signals and a second selected portion of the second discrete frequency signals,
wherein the first selected portion of the second discrete frequency signals is in a first frequency range of 15.625 Hz to 31.25 Hz, and
wherein the second selected portion of the second discrete frequency signals is in a second frequency range of 7.8125 Hz to 15.625 Hz;
select a first portion of the generated second power spectrum signal;
divide the selected first portion of the generated second power spectrum signal into one or more second subsets comprising sub-portions of the selected first portion of the generated second power spectrum signal;
for each subset in the one or more second subsets of the selected first portion of the generated second power spectrum signal:
compute a peak value of the sub-portion of the selected first portion of the second power spectrum signal falling within the subset; and
store the computed peak value of the subset in a second threshold buffer; and
compute a second adaptive threshold R-peak value based on the computed peak values stored in the second threshold buffer.

3. The system of claim 2 wherein the at least one controller is further configured to:
update the first adaptive threshold R-peak value based on the second adaptive threshold R-peak value.

4. The system of claim 2 wherein the at least one controller is further configured to:
determine whether the received second electrocardiogram signal includes a QRS complex by applying the computed first adaptive threshold R-peak value, or the computed second adaptive threshold R-peak value, or both to the generated second power spectrum signal;
when it is determined that the received electrocardiogram signal includes the QRS complex, compute an R-to-R time interval value indicative of timing between two successive R-peaks in successive QRS complexes in the received second electrocardiogram signal of the patient based on: the computed first adaptive threshold R-peak value, or the computed second adaptive threshold R-peak value, or both.

5. The system of claim 4 wherein the at least one controller is further configured to:
generate a prediction of timing of a future R-peak based on the computed R-to-R time interval value indicative of timing between two successive R-peaks in successive QRS complexes.

6. The system of claim 5 wherein the at least one controller is further configured to:
trigger an inflation of the at least one intra-aortic balloon pump based on the generated prediction of timing of the future R-peak.

7. The system of claim 1 further comprising:
at least one skin interface device configurably connected to the at least one intra-aortic balloon pump and configurably connected to the at least one controller,
wherein the at least one controller is further configured to receive the identity of the patient and a set of patient specific information from the at least one skin interface device.

8. The system of claim 1 wherein the at least one transformation signal is based on a Haar wavelet.

9. The system of claim 1 wherein the first portion of the generated first power spectrum signal spans 7.5 seconds.

10. The system of claim 2 wherein the first portion of the generated second power spectrum signal spans 7.5 seconds.

11. The system of claim 1 wherein each subset in the one or more subsets of the selected first portion of the generated first power spectrum signal spans 1.5 seconds.

12. The system of claim 2 wherein each subset in the one or more second subsets of the selected first portion of the generated second power spectrum signal spans 1.5 seconds.

13. The system of claim 1 wherein the one or more parameters comprise:
name of the patient,
age of the patient,
average heart rate of the patient,
maximum heart rate of the patient,
minimum heart rate of the patient,
pulse of the patient,
average R-to-R time interval of the patient,
maximum R-to-R time interval of the patient,
minimum R-to-R time interval of the patient,
average R-peak of the patient,
maximum R-peak of the patient,
minimum R-peak of the patient,
or any combination thereof.

14. A computer implemented method for operating intra-aortic balloon pumps, the method comprising:
initializing values of one or more parameters based on an identity of a patient;
receiving a first electrocardiogram signal of the patient;
generating a first power spectrum signal by decomposing the received first electrocardiogram signal, wherein the first power spectrum signal is generated by:
applying at least one transformation signal to decompose the received first electrocardiogram signal into discrete frequency signals;
selecting one or more portions of the discrete frequency signals; and
computing the first power spectrum signal by aligning the selected one or more portions of discrete frequency signals,
wherein the computed first power spectrum signal is based on a first selected portion of the discrete frequency signals and a second selected portion of the discrete frequency signals,
wherein the first selected portion of the discrete frequency signals is in a first frequency range of 15.625 Hz to 31.25 Hz, and
wherein the second selected portion of the discrete frequency signals is in a second frequency range of 7.8125 Hz to 15.625 Hz;
selecting a first portion of the generated first power spectrum signal;
dividing the selected first portion of the generated first power spectrum signal into one or more subsets comprising sub-portions of the selected first portion of the generated first power spectrum signal;
for each subset in the one or more subsets of the selected first portion of the generated first power spectrum signal:
computing a peak value of the sub-portion of the selected first portion of the first power spectrum signal falling within the subset; and
storing the computed peak value of the subset in a first threshold buffer; and
computing a first adaptive threshold R-peak value based on the computed peak values stored in the first threshold buffer.

15. The computer-implemented method of claim 14 further comprising:
receiving a second electrocardiogram signal of the patient;
generating a second power spectrum signal by decomposing the received second electrocardiogram signal, wherein the second power spectrum signal is generated by:
applying at least one transformation signal to decompose the received second electrocardiogram signal into second discrete frequency signals;
selecting one or more portions of the second discrete frequency signals; and
computing the second power spectrum signal by aligning the selected one or more portions of the second discrete frequency signals,
wherein the computed second power spectrum signal is based on a first selected portion of the second discrete frequency signals and a second selected portion of the second discrete frequency signals,
wherein the first selected portion of the second discrete frequency signals is in a first frequency range of 15.625 Hz to 31.25 Hz, and
wherein the second selected portion of the second discrete frequency signals is in a second frequency range of 7.8125 Hz to 15.625 Hz;
selecting a first portion of the generated second power spectrum signal;
dividing the selected first portion of the generated second power spectrum signal into one or more second subsets comprising sub-portions of the selected first portion of the generated second power spectrum signal;
for each subset in the one or more second subsets of the selected first portion of the generated second power spectrum signal:
computing a peak value of the sub-portion of the selected first portion of the second power spectrum signal falling within the subset; and
storing the computed peak value of the subset in a second threshold buffer; and
computing a second adaptive threshold R-peak value based on the computed peak values stored in the second threshold buffer.

16. The computer-implemented method of claim 15 further comprising:
updating the first adaptive threshold R-peak value based on the second adaptive threshold R-peak value.

17. The computer-implemented method of claim 15 further comprising:
determining whether the received second electrocardiogram signal includes a QRS complex by applying the computed first adaptive threshold R-peak value, or the computed second adaptive threshold R-peak value, or both to the generated second power spectrum signal; and
when it is determined that the received electrocardiogram signal includes the QRS complex, computing an R-to-R time interval value indicative of timing between two successive R-peaks in successive QRS complexes in the received second electrocardiogram signal of the patient based on: the computed first adaptive threshold R-peak value, or the computed second adaptive threshold R-peak value, or both.

18. A computer implemented method for operating intra-aortic balloon pumps, the method comprising:
initializing values of one or more parameters based on an identity of a patient;
receiving a first electrocardiogram signal of the patient;
generating a first power spectrum signal by decomposing the received first electrocardiogram signal, wherein the first power spectrum signal is generated by:
applying at least one transformation signal to decompose the received first electrocardiogram signal into discrete frequency signals;
selecting one or more portions of the discrete frequency signals; and
computing the first power spectrum signal by aligning the selected one or more portions of discrete frequency signals,
wherein the computed first power spectrum signal is based on a first selected portion of the discrete frequency signals and a second selected portion of the discrete frequency signals,
wherein the first selected portion of the discrete frequency signals is in a first frequency range of 15.625 Hz to 31.25 Hz, and
wherein the second selected portion of the discrete frequency signals is in a second frequency range of 7.8125 Hz to 15.625 Hz;
selecting a first portion of the generated first power spectrum signal;
dividing the selected first portion of the generated first power spectrum signal into one or more subsets comprising sub-portions of the selected first portion of the generated first power spectrum signal;
for each subset in the one or more subsets of the selected first portion of the generated first power spectrum signal:
computing a peak value of the sub-portion of the selected first portion of the first power spectrum signal falling within the subset; and
storing the computed peak value of the subset in a first threshold buffer;
computing a first adaptive threshold R-peak value based on the computed peak values stored in the first threshold buffer;
receiving a second electrocardiogram signal of the patient;
generating a second power spectrum signal by decomposing the received second electrocardiogram signal, wherein the second power spectrum signal is generated by:
applying at least one transformation signal to decompose the received second electrocardiogram signal into second discrete frequency signals;
selecting one or more portions of the second discrete frequency signals; and
computing the second power spectrum signal by aligning the selected one or more portions of the second discrete frequency signals,
wherein the computed second power spectrum signal is based on a first selected portion of the second discrete frequency signals and a second selected portion of the second discrete frequency signals,
wherein the first selected portion of the second discrete frequency signals is in a first frequency range of 15.625 Hz to 31.25 Hz, and
wherein the second selected portion of the second discrete frequency signals is in a second frequency range of 7.8125 Hz to 15.625 Hz;
selecting a first portion of the generated second power spectrum signal;
dividing the selected first portion of the generated second power spectrum signal into one or more second subsets comprising sub-portions of the selected first portion of the generated second power spectrum signal;
for each subset in the one or more second subsets of the selected first portion of the generated second power spectrum signal:
computing a peak value of the sub-portion of the selected first portion of the second power spectrum signal falling within the subset; and
storing the computed peak value of the subset in a second threshold buffer;
computing a second adaptive threshold R-peak value based on the computed peak values stored in the second threshold buffer;
computing an R-to-R time interval value indicative of timing between two successive R-peaks in successive QRS complexes in the received second electrocardiogram signal of the patient based on: the computed first adaptive threshold R-peak value, or the computed second adaptive threshold R-peak value, or both;
generating a prediction of timing of a future R-peak based on the computed R-to-R time interval value indicative of timing between two successive R-peaks in successive QRS complexes; and
triggering an inflation of at least one intra-aortic balloon pump based on the generated prediction of timing of the future R-peak.

19. The computer-implemented method of claim 18 further comprising:
updating the first adaptive threshold R-peak value based on the second adaptive threshold R-peak value.

20. The computer-implemented method of claim 18 wherein the one or more parameters comprise:
name of the patient,
age of the patient,
average heart rate of the patient,
maximum heart rate of the patient,
minimum heart rate of the patient,
pulse of the patient,
average R-to-R time interval of the patient,
maximum R-to-R time interval of the patient,
minimum R-to-R time interval of the patient,
average R-peak of the patient,
maximum R-peak of the patient,
minimum R-peak of the patient,
or any combination thereof.
